(19) United States
(12) Patent Application Publication
(10) Pub. No.: US 2022/0016679 A1
(43) Pub. Date: Jan. 20, 2022

(54) CONTINUOUS POSITIVE AIRWAY PRESSURE (CPAP) MACHINE CLEANING SYSTEM

(71) Applicant: David Alan Poole, Qualicum Beach, BC (CA)

(72) Inventor: David Alan Poole, Qualicum Beach, BC (CA)

(21) Appl. No.: 17/377,946

(22) Filed: Jul. 16, 2021

Related U.S. Application Data
(60) Provisional application No. 63/052,610, filed on Jul. 16, 2020.

(51) Int. Cl. B08B 5/02; B08B 9/032; B08B 9/023; A61L 2/20; A61L 2/26; A61M 16/08

(52) U.S. Cl. CPC B08B 5/02; A61M 2205/8206; B08B 9/023; A61L 2/202; A61L 2/26; A61M 16/0875; B08B 2209/032; A61L 2202/11; A61L 2202/24; A61L 2202/14; A61L 2202/15; A61L 2202/121; A61M 2209/10; A61M 2205/75; B08B 9/032

(57) ABSTRACT

A continuous positive airway pressure (CPAP) machine cleaning system. The system may include a lower enclosure with an ozone generator and a fan all located therewithin. The system may also include an upper enclosure with a dehumidifier mechanism housed within a rear portion thereof, a CPAP hose and CPAP mask connection located within the rear portion thereof, capable of removable attachment to a CPAP hose and a filter located on a top of thereof and capable of being removable therefrom.

### FIELD OF THE INVENTION

The present invention relates to a cleaning system for sanitizing Continuous Positive Airway Pressure (CPAP) machines.

### BACKGROUND OF THE INVENTION

Sleep apnea is a condition in which a patient's breathing passage seals during sleep, causing the patient to stop breathing. The typical response to this lack of oxygen is the arousing or waking from sleep. This cycle then repeats with the result of the patient being unable to get the needed quantity of restful sleep. This results in many side effects such as fatigue, difficulty performing mental tasks, and irritability. If left untreated, sleep apnea can result in heart attacks, strokes, high blood pressure, diabetes, and may even result in death. It is typically treated by Continuous Positive Airway Pressure (CPAP) therapy which provides a constant airflow through a nasal mask that typically seals around the nose and mouth.

Current CPAP machines, however, suffer from two distinct disadvantages. First, they are typically powered by AC power making them useless during a power failure, or when used at a sleeping location without AC power. Second, they require diligent cleaning to keep them free from bacteria, mold, and other contaminants. Accordingly, there exists a need for a means by which CPAP machines can be modified to address these two shortcomings. The development of the self-cleaning and self-powered continuous positive airway pressure machine fulfills this need.

### SUMMARY OF THE INVENTION

According to the present invention there is provided a continuous positive airway pressure (CPAP) machine cleaning system. The system may include a lower enclosure with an ozone generator and a fan all located therewithin. The system may also include an upper enclosure with a dehumidifier mechanism housed within a rear portion thereof, a CPAP hose and CPAP mask connection located within the rear portion thereof, capable of removable attachment to a CPAP hose and a filter located on a top of thereof and capable of being removable therefrom.

The lower enclosure may include a battery located therewithin.

The upper enclosure may have a power switch located on a front face thereof in electrical communication with the battery, a control knob located on the front face thereof in electrical communication with the power switch, a display screen located on the front face thereof in electrical communication with the control knob, a data access door located on a side face thereof, a two-part access door located on the rear portion thereof.

During a cleaning operation, the fan may generate a forced flow of air through said ozone generator to generate an ozone air flow and an air flow bath. The ozone air flow may be directed towards the CPAP hose and CPAP mask connection and the air flow bath may be directed to the filter.

Other aspects of the invention will be appreciated by reference to the detailed description of the preferred embodiment and to the claims that follow.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a front perspective view of the CPAP machine cleaning system, according to the preferred embodiment of the present invention;

FIG. 2 is a rear perspective view of the CPAP machine cleaning system, according to the preferred embodiment of the present invention;

FIG. 3 is a sectional view of the CPAP machine cleaning system, as seen along a line I-I, as shown in FIG. 1, according to the preferred embodiment of the present invention;

FIG. 4 is a sectional view of the CPAP machine cleaning system, as seen along a line II-II, as shown in FIG. 1, according to the preferred embodiment of the present invention;

FIG. 5 is an electrical block diagram of the CPAP machine cleaning system, according to the preferred embodiment of the present invention; and

FIG. 6 is perspective view of a power strip of the invention.

### DESCRIPTION OF THE PREFERRED EMBODIMENT OF THE INVENTION

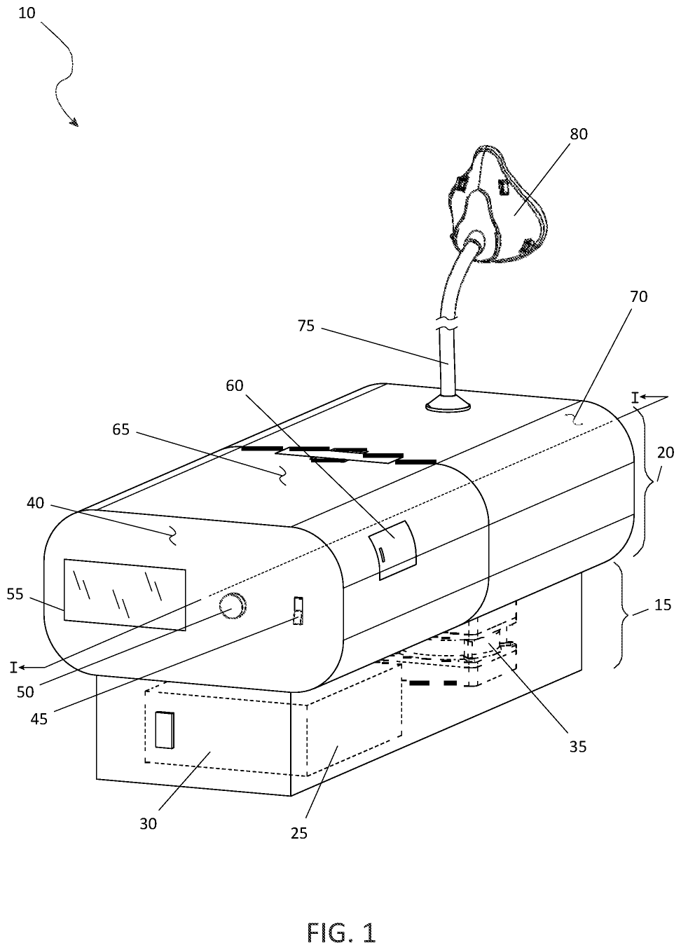

The best mode for carrying out the invention is presented in terms of its preferred embodiment, herein depicted within FIGS. 1 through 4. However, the invention is not limited to the described embodiment, and a person skilled in the art will appreciate that many other embodiments of the invention are possible without deviating from the basic concept of the invention and that any such work around will also fall under scope of this invention. It is envisioned that other styles and configurations of the present invention can be easily incorporated into the teachings of the present invention, and only one (1) particular configuration shall be shown and described for purposes of clarity and disclosure and not by way of limitation of scope. The implementations described below are exemplary implementations provided to enable persons skilled in the art to make or use the embodiments of the disclosure and are not intended to limit the scope of the disclosure, which is defined by the claims.

The terms “a” and “an” herein do not denote a limitation of quantity, but rather denote the presence of at least one (1) of the referenced items.

Referring now to FIG. 1, a front perspective view of the CPAP machine cleaning system 10, according to the preferred embodiment of the present invention is disclosed. The CPAP machine cleaning system 10 (herein also described as the “machine”), provides for a CPAP machine that can be self-powered for multiple days and is self-cleaning. The machine 10 provides for a lower enclosure 15 and an upper enclosure 20 as shown. The lower enclosure 15 houses a rechargeable battery 25 (shown by dashed lines due to its hidden state) behind a battery door 30. The battery door 30 allows access to the battery 25 for recharging and replacement. An ozone generator 35 (shown by dashed lines due to its hidden state) is located in the rear of the lower enclosure 15. Further description of both the rechargeable battery 25 and the ozone generator 35 will be provided herein below.

The upper enclosure 20 houses conventional components most often found with a conventional CPAP machine. A front face 40 provides for a power switch 45, a control knob 50 and a display screen 55, all of which operate in a normal manner. The side of the upper enclosure 20 provides a data access door 60 which covers electronic ports that allow the CPAP mechanism 65 to be adjusted to an individual patient's needs as well as recovering operating data to allow for further refinements in treatment. The rear portion of the upper enclosure 20 houses a dehumidifier mechanism 70, as well as allowing for connection to a CPAP hose 75 and a CPAP mask 80. The presence or omission of other controls, accessories, or features of available CPAP machines is not intended to be a limiting feature of the present invention.

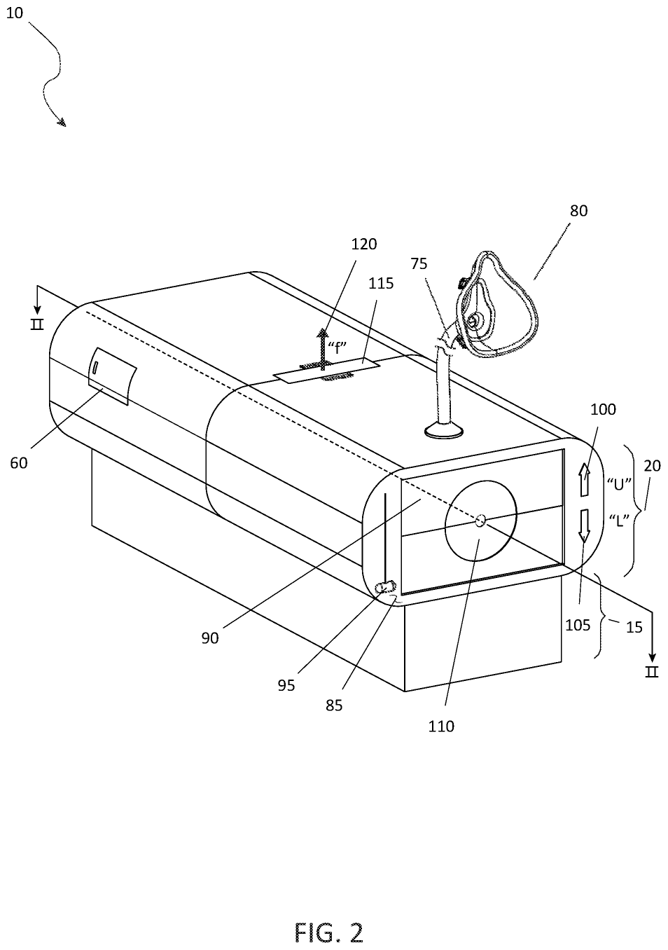

Referring next to FIG. 2, a rear perspective view of the machine 10, according to the preferred embodiment of the present invention is depicted. This view provides further clarification on the configuration of the lower enclosure 15 and the upper enclosure 20. The data access door 60 is visible on the side of the upper enclosure 20. The rear face 85 of the upper enclosure 20 is provided with a two-part access door 90 that opens via a sliding lever 95 along an upper travel path “u” 100 and a lower travel path “I” 105. A circular air-tight gasket 110 seals around the CPAP hose 75 while the CPAP mask 80 is placed inside of the two-part access door 90. A removable filter 115 is located on the top of the upper enclosure 20 and is removed by pulling it upward along a filter removal path “f” 120.

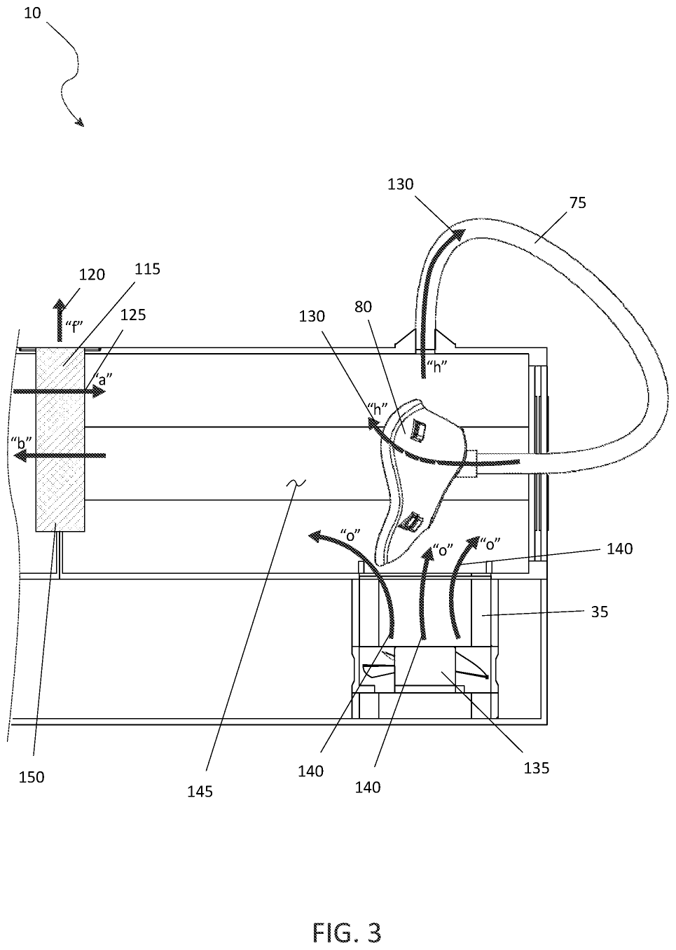

Referring now to FIG. 3, a sectional view of the machine 10, as seen along a line I-I, as shown in FIG. 1, according to the preferred embodiment of the present invention is shown. The removable filter 115 is again shown along with the filter removal path “f” 120. During operation of the CPAP mechanism 65, air is moved through the removable filter 115 following a first air flow path “a” 125, whereupon it enters the CPAP hose 75 and exits through the CPAP mask 80 while following a hose air flow path “h” 130. During cleaning operation, a fan 135 propels air through the ozone generator 35, thus generating an ozone air flow path flow “o” 140 into a plenum space 145. During the same cleaning operation, the ozone air flow path flow “o” 140 thus generates a second air flow bath “b” 150 through the removable filter 115 to clean it. Likewise, the ozone air flow path flow “o” 140 also enters the CPAP hose 75 via the hose air flow path “h” 130 and the CPAP mask 80 via the hose air flow path “h” 130 thus cleaning and sanitizing them as well.

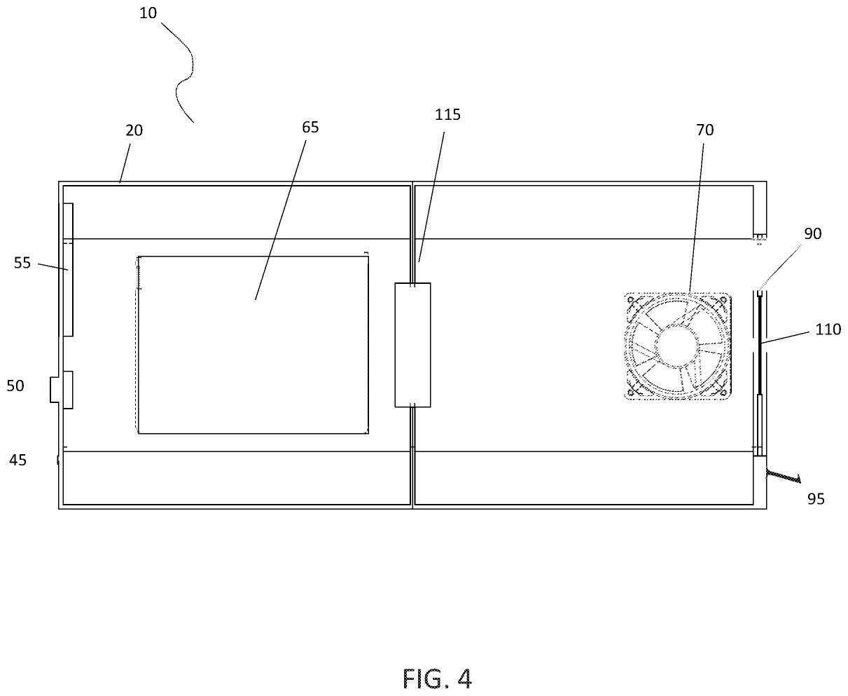

Referring next to FIG. 4, a sectional view of the machine 10, as seen along a line II-II, as shown in FIG. 1, according to the preferred embodiment of the present invention is disclosed. The CPAP mechanism 65 is located on one (1) side of the upper enclosure 20 along with the power switch 45, the control knob 50, and the display screen 55. The removable filter 115 separates the CPAP mechanism 65 from the dehumidifier mechanism 70. The rear of the upper enclosure 20 provides for the two-part access door 90, operated by the sliding lever 95, and the gasket 110.

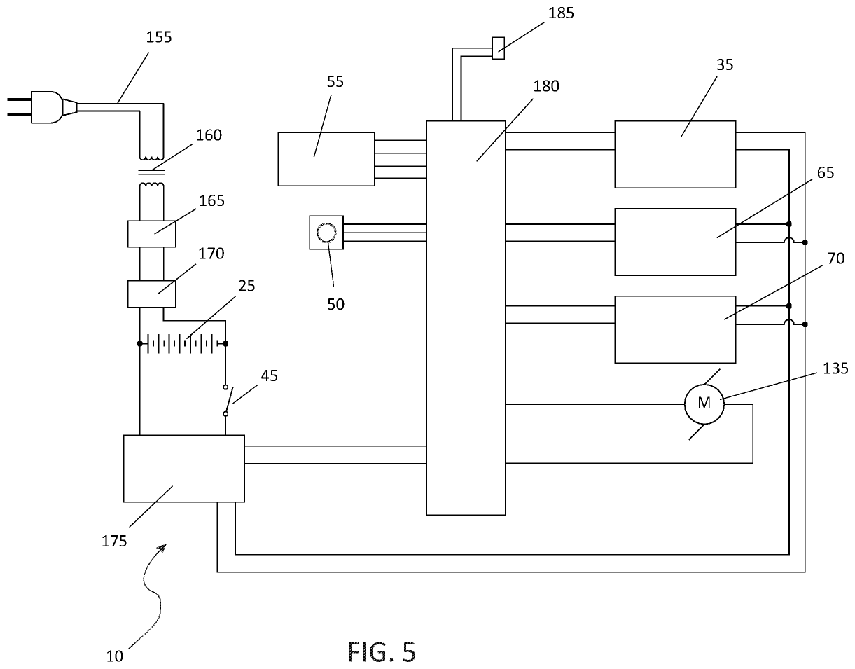

Referring to FIG. 5, an electrical block diagram of the machine 10, according to the preferred embodiment of the present invention is depicted. An AC power cord 155 brings incoming electrical power to a step-down transformer 160 and a rectifier 165 which produces low voltage DC power. The resultant power flows to a charge controller 170 which maintains charge on the rechargeable battery 25 whenever the AC power cord 155 is connected to a source of AC power. The power switch 45, when closed, passes DC power to a power supply 175. Conditioned power then flows to a main controller 180 such as a single board computer (SBC) running a dedicated operating system. The main controller 180 receives inputs from the display screen 55 and the control knob 50, while providing outputs to the ozone generator 35, the CPAP mechanism 65, the dehumidifier mechanism 70, and the fan 135. The power supply 175 simultaneously provides high current power to the ozone generator 35, the CPAP mechanism 65, and the dehumidifier mechanism 70 as well. It is envisioned that the rechargeable battery 25 has a capacity to allow operation of the machine 10 for a period of up to forty-eight hours. Additionally, it is envisioned that the cleaning cycle using the ozone generator 35 will run for a period of four minutes as controlled by the main controller 180. Finally, a data access port 185, which is located behind the data access door 60 (as shown in FIG. 1), allows for both downloaded and uploading of pertinent operating data to allow for customized usage for individual users.

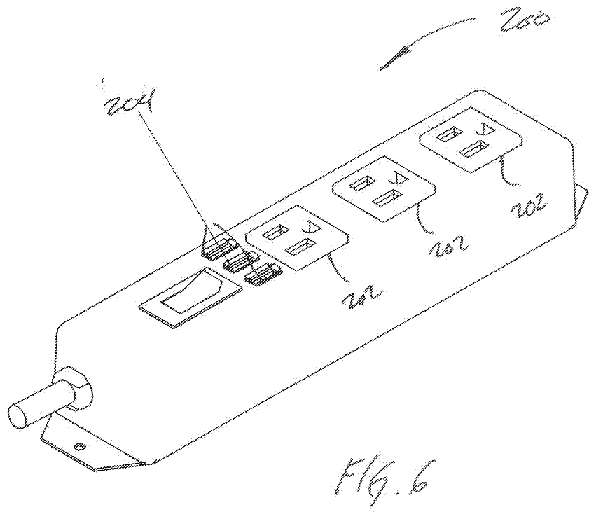

The AC outlet power charges the rechargeable battery 25 that is connected to a customizable power strip 200. As depicted in FIG. 6, the power strip 200 includes three AC plugins 202 with ground and will also include three USB type C ports 204. The power strip 200 also has its own AC plug for direct power from the AC outlet. This will permit the components to run directly from the outlet without the rechargeable battery 25.

The CPAP machine 10 is equipped with the rechargeable battery 25 that plugs in directly to a United States AC outlet. The rechargeable battery 25 is connected and is central to the entire power management system so, in the event of a power outage or a shortage of electricity in the circuit the rechargeable battery 25 serves as a backup component to keep the entire unit operational. The rechargeable battery 25 is removable. With the redistribution of cords within the design, the power strip 200 can plug in directly to an AC outlet to power machine operations. This can be done to reduce the weight of the machine 10 or to prevent an unnecessary component for a customers' inconvenience. Finally, a buck step-down converter can be used to handle power supplies used, in addition to the removal of the rechargeable battery 25.

Referring to FIG. 6, the power strip 200 distributes power to a two-part PCB design and other particular components including the ozone generator 35 dehumidifier mechanism, an atomizer, a relay switch, a mini air compressor, and an air actuated valve. The user can control and activate the machine 10 through a touch screen at the front of the machine 10. The machine 10 automatically regulates air pressure through the touch screen and through the servo motor that is equipped to the plastic ball valve. The user can assign a certain percentage value to the valve to control the amount of opening that is present in the ball valve. The ball valve opening, and closing will create pressure differences that is controlled through the control nob and this metric can be studied and displayed on the touch screen.

Air pressure control will be achieved by using a mini air compressor in conjunction with an air actuated valve that contains a small servo motor. The servo motor and the wiring thereof, will be attached to the PCB design with a 10K potentiometer. This will allow the control of power and movement to the servo arm which in turn will allow the ball valve to be controlled by the user. As the ball valve is toggled by the potentiometer control nob it will create a digital readout on the touch screen at the front of the design. Accompanying the unit will be a digital sensor or gauge that will be in the pipeline of the hose to properly gauge real time air pressures as the machine is running. The digital sensor will be able to send the air pressure in PSI directly to the touch screen for the users' awareness.

A filter box with the help of the mini air compressor will push the ozone gas in the lower enclosure. The flow of ozone will continue through the hose 75 and through the mask 80 to allow it to be deodorized. This ozone release feature can be turned off through the special safety settings in the built-in design timer. And azone dispensing can be controlled through the touch screen 55 and PCB design). This protects the user from being exposed to harmful ozone gas while using the CPAP 10 function to assist in breathing.

The air hose 75 for the CPAP 10 will be routed from the air actuated valve through a compartment wall. It then will travel through the divider between the upper 20 and lower 15 enclosures and finally routed up to the filter box. The filter box will have exposure to the ozone through the machine's 10 normal operation. When in the cleaning cycle the unit is equipped with HEPA filters on the side, top, and around the upper enclosure 20 and filter. This is to prevent the buildup of mold, dust, pollen and or volatile organic compounds (VOC) in breathing air supply.

Humidification will be achieved through a PCB controlled design using an atomizer. The atomizer's purpose is to provide small amounts of vibration to project small molecules of water into the atmosphere. This is to create a humid environment without the use of heat. The atomizer will be the chief source of creating a humid environment within the breathing mask 80 and hose 75.

A user may take the mask 80 from the CPAP machine 10 and inserts it through flaps in the upper enclosure 20. After hose 75 and mask 80 are safely inserted through the flaps a protective wall can be deactivated by lowering a bar. Opening the device can also be equipped with a Magnetic Hall Sensor and a magnet to detect by PCB if a trap door has been opened.

### Operation of the Preferred Embodiment

The preferred embodiment of the present invention can be utilized by the common user in a simple and effortless manner with little or no training. It is envisioned that the self-cleaning and machine 10 would be constructed in general accordance with FIGS. 1 through 5. The user would procure the machine 10 from conventional procurement channels such as medical supply houses, doctor's offices, pharmaceutical supply houses, and the like. After procurement and prior to utilization, the self-cleaning and self-powered continuous positive airway pressure machine 10 would be prepared in the following manner: the machine 10 would be located near a sleeping location; the AC power cord 155 would be connected to a suitable source of AC power if available, otherwise the machine 10 will operate on the battery 25; customized usage parameters would be downloaded by the data access port 185, and a removable filter 115 would be inserted into the lower enclosure 15. At this point in time, the machine 10 is ready for operation.

During utilization of the self-cleaning and self-powered continuous positive airway pressure machine 10 as a standard CPAP, the user would follow conventional operating procedures which are not limited by the present invention. should AC power be lost during operation, the parallel configuration of the battery 25 (as shown in FIG. 5) allows for continuous uninterrupted operation.

After use of the CPAP machine cleaning system 10, the CPAP mask 80, with the CPAP hose 75 remaining attached, is inserted into the two-part access door 90, which is opened via the sliding lever 95; the user, via the display screen 55 initiates a four minute cleaning mode in which ozone is passed through the removable filter 115, the CPAP hose 75 and the CPAP mask 80 to kill any lingering germs and bacteria. The enclosed arrangement and sealing of the CPAP hose 75 via the gasket 110 prevents potentially hazardous ozone from escaping the lower enclosure 15 or the upper enclosure 20 and posing a hazard to anyone present nearby. At the completion of the four minute cleaning cycle, the CPAP mask 80 may remain inside the two-part access door 90 until needed again for the CPAP functionality of the machine 10. This process then continues in a repeating cyclic manner.

The ability of to the machine 10 to operate without AC electric power via the AC power cord 155 makes the machine 10 ideal for use during power failures, at remote locations, or when sleeping in a mobile environment such as on a train, boat, a recreational vehicle, or when camping.

The self-cleaning functionality of the ozone generator 35 provided by the machine 10 ensures that usage of the machine 10 will not sicken or otherwise negatively impact the health of the user while providing a clean and fresh smelling experience.

The foregoing descriptions of specific embodiments of the present invention have been presented for purposes of illustration and description. They are not intended to be exhaustive or to limit the invention to the precise forms disclosed, and obviously many modifications and variations are possible in-light-of the above teaching. The embodiments were chosen and described in order to best explain the principles of the invention and its practical application, to thereby enable others skilled in the art to best utilize the invention and various embodiments with various modifications as are suited to the particular use contemplated.

What is claimed is:

1. A continuous positive airway pressure (CPAP) machine cleaning system, comprising:
a lower enclosure, having an ozone generator, and a fan all located therewithin;
an upper enclosure, having:
a dehumidifier mechanism housed within a rear portion thereof;
a CPAP hose and CPAP mask connection located within said rear portion thereof, capable of removable attachment to a CPAP hose;
a filter is located on a top of thereof and capable of being removable therefrom;
wherein during a cleaning operation, said fan generates a forced flow of air through said ozone generator to generate an ozone flow and an air flow bath;
wherein said ozone air flow is directed towards said CPAP hose and CPAP mask connection; and
wherein said air flow bath is directed to said filter.

2. The continuous positive airway pressure (CPAP) machine cleaning system of claim 1 wherein said lower enclosure further comprises a battery located therewithin.

3. The continuous positive airway pressure (CPAP) machine cleaning system of claim 2 wherein said upper enclosure further comprises:
a power switch located on a front face thereof in electrical communication with said battery;
a control knob located on said front face thereof in electrical communication with said power switch;
a display screen located on said front face thereof in electrical communication with said control knob;
a data access door located on a side face thereof; and
a two-part access door located on said rear portion thereof.

4. A continuous positive airway pressure (CPAP) machine cleaning system, comprising:
a lower enclosure, having a battery, an ozone generator, and a fan all located therewithin;
an upper enclosure, having:
a power switch located on a front face thereof in electrical communication with said battery;
a control knob located on said front face thereof in electrical communication with said power switch;
a display screen located on said front face thereof in electrical communication with said control knob;
a data access door located on a side face thereof;
a dehumidifier mechanism housed within a rear portion thereof in electrical communication with said control knob;
a CPAP hose and CPAP mask connection located within said rear portion thereof, capable of removable attachment to a CPAP hose;
a two-part access door located on said rear portion thereof; and
a filter is located on a top of thereof and capable of being removable therefrom;
wherein during a cleaning operation, said fan generates a forced flow of air through said ozone generator to generate an ozone flow and an air flow bath;
wherein said ozone air flow is directed towards said CPAP hose and CPAP mask connection; and
wherein said air flow bath is directed to said filter.
